(19) United States
(12) Patent Application Publication
(10) Pub. No.: US 2020/0000861 A1
(43) Pub. Date: Jan. 2, 2020

(54) TREATMENT OF CANCER

(71) Applicant: Virttu Biologics Limited, Newhouse (GB)

(72) Inventors: Joe Conner, Newhouse (GB); Timothy Cripe, Newhouse (GB)

(73) Assignee: Virttu Biologics Limited, Newhouse (GB)

(21) Appl. No.: 16/447,760

(22) Filed: Jun. 20, 2019

Related U.S. Application Data
(63) Continuation of application No. PCT/EP2017/084421, filed on Dec. 22, 2017.

(30) Foreign Application Priority Data
Dec. 23, 2016 (GB) 1622214.3
Feb. 17, 2017 (GB) 1702565.1

(51) Int. Cl. A61K 35/763; A61K 9/00; A61P 35/00

(52) U.S. Cl. CPC A61K 35/763; A61K 45/06; A61P 35/00; A61K 9/0019

(57) ABSTRACT

Oncolytic herpes simplex virus for use in a method of treating cancer in a pediatric subject having a tumor are described, wherein the oncolytic herpes simplex virus is administered intratumorally.

This application claims priority from GB 1622214.3 filed 23 Dec. 2016 and from GB 1702565.1 filed 17 Feb. 2017, the contents and elements of which are herein incorporated by reference for all purposes.

### FIELD OF THE INVENTION

The present invention relates to the use of an oncolytic herpes simplex virus in the treatment of cancer.

### BACKGROUND

Oncolytic virotherapy concerns the use of lytic viruses which selectively infect and kill cancer cells. Some oncolytic viruses are promising therapies as they display exquisite selection for replication in cancer cells and their self-limiting propagation within tumors results in fewer toxic side effects. Several oncolytic viruses have shown great promise in the clinic (Bell, J., Oncolytic Viruses: An Approved Product on the Horizon? Mol Ther. 2010; 18(2): 233-234).

### SUMMARY OF THE INVENTION

In one aspect an oncolytic herpes simplex virus for use in a method of treating cancer in a human pediatric subject having a tumor is provided, wherein the oncolytic herpes simplex virus is administered intratumorally.

In one aspect a method of treating cancer in a human pediatric subject is provided, the method comprising administering an oncolytic herpes simplex virus to a pediatric subject having a tumor, wherein the oncolytic herpes simplex virus is administered intratumorally.

In one aspect the use of an oncolytic herpes simplex virus in the manufacture of a medicament for use in a method of treating cancer in a human pediatric subject is provided, the method comprising administering an oncolytic herpes simplex virus to a pediatric subject having a tumor, wherein the oncolytic herpes simplex virus is administered intratumorally.

The oncolytic herpes simplex virus may be administered by intratumoral injection.

The tumor may be a solid tumor.

The oncolytic herpes simplex virus may be administered by image guided injection.

The method of treatment may comprise simultaneous, sequential or separate administration with a cytotoxic or cytostatic agent, an immunomodulatory agent, or radiation therapy.

The method may comprise determining the level of Treg cells in the subject prior to treatment with oncolytic herpes simplex virus, during a course of treatment with oncolytic herpes simplex virus and/or following conclusion of a course of treatment with oncolytic herpes simplex virus.

The method may comprise simultaneous, sequential or separate administration of an agent that supresses the regulatory T cell (Treg) response or population in the subject.

The method may comprise determining pseudoprogression of the tumor prior to treatment with oncolytic herpes simplex virus, during a course of treatment with oncolytic herpes simplex virus and/or following conclusion of a course of treatment with oncolytic herpes simplex virus.

In one aspect a method of selecting a human subject for continued treatment with an oncolytic herpes simplex virus is provided, the method comprising detecting a change in metabolic activity of a tumor in a human subject following administration of oncolytic herpes simplex virus to the subject, selecting a subject in which a change is detected to receive further administration of oncolytic herpes simplex virus.

Detecting a change in metabolic activity may involve detecting pseudoprogression.

The change in metabolic activity may be an increase in metabolic activity.

The change in metabolic activity or detection of pseudoprogession may be detected by imaging the tumor, e.g. using positron emission tomography (PET) and a suitably labelled metabolically active contrast agent such as ¹⁸F-deoxyglucose, computer tomography (CT) scanning or magnetic resonance imaging (MRI). Tumor imaging and detection of changes in metabolic activity or pseudoprogression may be determined by conducting the detection (e.g. imaging) more than once at different time points before, during and/or after a course of treatment with oncolytic herpes simplex virus.

Tumor pseudoprogression can manifest as an increase of lesion size related to treatment, which simulates progressive disease. The increase may be transient. Pseudoprogression can occur during immunotherapy treatments where initial imaging of the tumor suggests progression, e.g. through increased metabolic activity or size, whereas prolonged monitoring shows good response of the tumor to treatment. The phenomenon is further discussed in Parvez K, Parvez A, Zadeh G. The Diagnosis and Treatment of Pseudoprogression, Radiation Necrosis and Brain Tumor Recurrence. International Journal of Molecular Sciences. 2014; 15(7):11832-11846. doi:10.3390/ijms150711832, and Brandes et al., Neuro-Oncology, Volume 10, Issue 3, 1 Jun. 2008, Pages 361-367.

The administration of oncolytic herpes simplex virus to the subject may be by intratumoral administration. The administration of oncolytic herpes simplex virus to the subject may be by intratumoral injection. The subject may be a pediatric subject.

The tumor may be a solid tumor.

The following paragraphs contain statements of broad combinations of the aspects and embodiments herein disclosed:—

A method of treating cancer in a pediatric subject, the method comprising administering an oncolytic herpes simplex virus to a pediatric subject having a tumor, wherein the oncolytic herpes simplex virus is administered by intratumoral injection.

An oncolytic herpes simplex virus for use in a method of treating cancer in a pediatric subject having a tumor, wherein the oncolytic herpes simplex virus is administered by intratumoral injection.

Use of an oncolytic herpes simplex virus in the manufacture of a medicament for use in a method of treating cancer in a pediatric subject having a tumor, wherein the oncolytic herpes simplex virus is administered by intratumoral injection.

The oncolytic herpes simplex virus may be administered by image guided injection, e.g. computer tomography-guided injection.

The method of treatment may further comprise simultaneous, sequential or separate administration with a cytotoxic or cytostatic agent, an immunomodulatory agent, or radiation therapy.

The method may comprise the step of determining the level of Treg cells in the subject prior to treatment with oncolytic herpes simplex virus, during a course/programme of treatment with oncolytic herpes simplex virus and/or following conclusion of a course/programme of treatment with oncolytic herpes simplex virus. The determination may involve analysis of a sample, e.g. of blood, serum or plasma, obtained from the subject. Determination of the level of Treg cells, e.g. determining a reduction of Treg cells, in response to treatment with oncolytic herpes simplex virus and/or accompanying chemotherapy or radiotherapy may be used to select the subject for continued treatment with oncolytic herpes simplex virus and/or accompanying chemotherapy or radiotherapy. Methods of determining Treg cells are well known in the art, e.g. see Collison L W, Vignali D A A. In Vitro Treg Suppression Assays. Methods in molecular biology (Clifton, N.J.). 2011; 707:21-37; Clark et al. Toxicol Pathol. 2012; 40(1):107-12. Epub 2011 Oct. 27.

The method of treatment may further comprise suppression of the regulatory T cell (Treg) response or population in the subject.

The method of treatment may further comprise simultaneous, sequential or separate administration of an agent that supresses the regulatory T cell (Treg) response or population in the subject.

An agent that supresses the regulatory T cell (Treg) response or population may be a chemotherapy agent, e.g. drug, or radiation therapy.

A method comprising detecting metabolic activity of a tumor in a subject following administration of oncolytic herpes simplex virus to the subject.

The method may be a method of determining the response of the subject to treatment with the oncolytic herpes simplex virus. The method may form part of a method of treatment of a cancer. The method of treatment may comprise intratumoral injection of oncolytic herpes simplex virus to a tumor in the subject.

The subject may be a pediatric subject.

The method may comprises detection of a change in the metabolic activity of a tumor. The change may be an increase in metabolic activity. The tumor may be a tumor to which oncolytic herpes simplex virus has been administered by intratumoral injection. Additionally, or alternatively, it may be a tumor to which oncolytic herpes simplex virus has not been directly administered, e.g. by intratumoral injection.

The metabolic activity may represent cell metabolism, inflammation, viral replication or cell death at/around the tumor/site of detection.

Detection of metabolic activity of a tumor is possible using imaging techniques known to those of ordinary skill in the art, e.g. using positron emission tomography (PET) and a suitably labelled metabolically active contrast agent such as ¹⁸F-deoxyglucose, computer tomography (CT) scanning or magnetic resonance imaging (MRI).

Detection of metabolic activity may be conducted before and/or after administration of oncolytic herpes simplex virus. Transient changes in metabolic activity following administration of oncolytic herpes simplex virus may be consistent with a biological, e.g. immune, response to the treatment and may indicate that the subject is suitable to receive further treatment with oncolytic herpes simplex virus.

As such, a method of selecting a patient for continued treatment with oncolytic herpes simplex virus is provided, the method comprising detecting a change in metabolic activity of a tumor in a subject following administration of oncolytic herpes simplex virus to the subject, e.g. by intratumoral injection, selecting a subject in which a change, e.g. increase, is detected to receive further administration of oncolytic herpes simplex virus.

The invention includes the combination of the aspects and preferred features described except where such a combination is clearly impermissible or expressly avoided.

### BRIEF DESCRIPTION OF THE FIGURES

Embodiments and experiments illustrating the principles of the invention will now be discussed with reference to the accompanying figures in which:

FIGS. 1A and 1B. Inflammatory reactions following virus injection as detected by PET/CT. Baseline images, needle tracks and injection sites (arrows), and follow up scans are shown for two patients who experienced a transient increase in SUV uptake following virus injection that ultimately returned near baseline. Although initially interpreted as tumor progression, in retrospect the spontaneous decrease suggests the uptake was due to a transient inflammatory reaction to virus (pseudoprogression). (A) Patient HSV06. The tumor mass is outlined in white, C=Cycle, D=Day. Notice the area of uptake drops to zero, suggesting tumor necrosis in the exact geographic distribution of the uptake. (B) Patient HSV08. Notice the pleural effusion (white arrows) that developed coincident with the increased PET signal, both of which spontaneously resolved. In addition to the injected right chest wall lesion, the uninjected left hilar lesion also showed a transient increase in PET signal suggesting a systemic effect.

FIG. 2. Table 1 showing patient diagnosis, age, prior chemotherapy regimens, previous radiation therapy, time from diagnosis to treatment, disease at trial entry, dose of HSV1716 administered and location of injected tumor.

FIG. 3. Table 2 showing patient serologic responses to single dose of intratumoral HSV1716.

FIG. 4. Table 3 showing adverse events possibly, probably or definitely attributable to intratumoral HSV1716 administration.

FIG. 5. Table 4 showing disease response and PET SUV changes relative to baseline in each injected tumor after each dose of intratumoral HSV1716.

FIG. 6. Table 5 showing disease response and PET SUV change relative to baseline after single dose of intratumoral HSV1716 in non-injected target lesions.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

### Oncolytic Herpes Simplex Virus

An oncolytic virus is a virus that will lyse cancer cells (oncolysis), preferably in a preferential or selective manner. Viruses that selectively replicate in dividing cells over non-dividing cells are often oncolytic. Oncolytic viruses are well known in the art and are reviewed in Molecular Therapy Vol. 18 No. 2 Feb. 2010 pg 233-234.

The herpes simplex virus (HSV) genome comprises two covalently linked segments, designated long (L) and short (S). Each segment contains a unique sequence flanked by a pair of inverted terminal repeat sequences. The long repeat (RL or R_{L}) and the short repeat (RS or R_{S}) are distinct.

The HSV ICP34.5 (also called γ34.5) gene, which has been extensively studied, has been sequenced in HSV-1 strains F and syn17+ and in HSV-2 strain HG52. One copy of the ICP34.5 gene is located within each of the RL repeat regions. Mutants inactivating one or both copies of the ICP34.5 gene are known to lack neurovirulence, i.e. be avirulent/non-neurovirulent (non-neurovirulence is defined by the ability to introduce a high titre of virus (approx. 10⁶ plaque forming units (pfu)) to an animal or patient without causing a lethal encephalitis such that the LD50 in animals, e.g. mice, or human patients is in the approximate range of ≥10⁶ pfu), and be oncolytic.

Preferred oncolytic Herpes Simplex Virus (oHSV) are replication-competent virus, being replication-competent at least in the target tumor/cancer cells.

Oncolytic HSV that may be used in the present invention include HSV in which one or both of the γ34.5 (also called ICP34.5) genes are modified (e.g. by mutation which may be a deletion, insertion, addition or substitution) such that the respective gene is incapable of expressing, e.g. encoding, a functional ICP34.5 protein. Preferably, in HSV according to the invention both copies of the γ34.5 gene are modified such that the modified HSV is not capable of expressing, e.g. producing, a functional ICP34.5 protein.

In some embodiments the oncolytic herpes simplex virus may be an ICP34.5 null mutant where all copies of the ICP34.5 gene present in the herpes simplex virus genome (two copies are normally present) are disrupted such that the herpes simplex virus is incapable of producing a functional ICP34.5 gene product. In other embodiments the oncolytic herpes simplex virus may lack at least one expressible ICP34.5 gene. In some embodiments the herpes simplex virus may lack only one expressible ICP34.5 gene. In other embodiments the herpes simplex virus may lack both expressible ICP34.5 genes. In still other embodiments each ICP34.5 gene present in the herpes simplex virus may not be expressible. Lack of an expressible ICP34.5 gene means, for example, that expression of the ICP34.5 gene does not result in a functional ICP34.5 gene product.

Oncolytic herpes simplex virus may be derived from any HSV including any laboratory strain or clinical isolate (non-laboratory strain) of HSV. In some preferred embodiments the HSV is a mutant of HSV-1 or HSV-2. Alternatively the HSV may be an intertypic recombinant of HSV-1 and HSV-2. The mutant may be of one of laboratory strains HSV-1 strain 17, HSV-1 strain F or HSV-2 strain HG52. The mutant may be of the non-laboratory strain JS-1. Preferably the mutant is a mutant of HSV-1 strain 17. The herpes simplex virus may be one of HSV-1 strain 17 mutant 1716, HSV-1 strain F mutant R3616, HSV-1 strain F mutant G207, HSV-1 mutant NV1020, or a further mutant thereof in which the HSV genome contains additional mutations and/or one or more heterologous nucleotide sequences. Additional mutations may include disabling mutations, which may affect the virulence of the virus or its ability to replicate. For example, mutations may be made in any one or more of ICP6, ICP0, ICP4, ICP27. Preferably, a mutation in one of these genes (optionally in both copies of the gene where appropriate) leads to an inability (or reduction of the ability) of the HSV to express the corresponding functional polypeptide. By way of example, the additional mutation of the HSV genome may be accomplished by addition, deletion, insertion or substitution of nucleotides.

A number of oncolytic herpes simplex viruses are known in the art. Examples include HSV1716, R3616 (e.g. see Chou & Roizman, Proc. Natl. Acad. Sci. Vol. 89, pp. 3266-3270, April 1992), G207 (Toda et al, Human Gene Therapy 9:2177-2185, Oct. 10, 1995), NV1020 (Geevarghese et al, Human Gene Therapy 2010 September; 21(9):1119-28), RE6 (Thompson et al, Virology 131, 171-179 (1983)), and Oncovex™ (Simpson et al, Cancer Res 2006; 66:(9) 4835-4842 May 1, 2006; Liu et al, Gene Therapy (2003): 10, 292-303), dlsptk, hrR3, R4009, MGH-1, MGH-2, G474, Myb34.5, DF3γ34.5, HF10, NV1042, RAMBO, rQNestin34.5, R5111, R-LM113, CEAICP4, CEAγ34.5, DF3γ34.5, KeM34.5 (Manservigi et al, The Open Virology Journal 2010; 4:123-156), rRp450, M032 (Campadelli-Fiume et al, Rev Med. Virol 2011; 21:213-226), Baco1 (Fu et al, Int. J. Cancer 2011; 129(6):1503-10) and M032 and C134 (Cassady et al, The Open Virology Journal 2010; 4:103-108).

In some preferred embodiments the herpes simplex virus is HSV-1 strain 17 mutant 1716 (HSV1716). HSV 1716 is an oncolytic, non-neurovirulent HSV and is described in EP 0571410, WO 92/13943, Brown et al (Journal of General Virology (1994), 75, 2367-2377) and MacLean et al (Journal of General Virology (1991), 72, 631-639). HSV 1716 has been deposited on 28 Jan. 1992 at the European Collection of Animal Cell Cultures, Vaccine Research and Production Laboratories, Public Health Laboratory Services, Porton Down, Salisbury, Wiltshire, SP4 0JG, United Kingdom under accession number V92012803 in accordance with the provisions of the Budapest Treaty on the International Recognition of the Deposit of Microorganisms for the Purposes of Patent Procedure (herein referred to as the ‘Budapest Treaty’). In some embodiments the herpes simplex virus is a mutant of HSV-1 strain 17 modified such that both ICP34.5 genes do not express a functional gene product, e.g. by mutation (e.g. insertion, deletion, addition, substitution) of the ICP34.5 gene, but otherwise resembling or substantially resembling the genome of the wild type parent virus HSV-1 strain 17+. That is, the virus may be a variant of HSV1716, having a genome mutated so as to inactivate both copies of the ICP34.5 gene of HSV-1 strain 17+ but not otherwise altered to insert or delete/modify other protein coding sequences.

In some embodiments the genome of an oncolytic Herpes Simplex Virus according to the present invention may be further modified to contain nucleic acid encoding at least one copy of a polypeptide that is heterologous to the virus (i.e. is not normally found in wild type virus) such that the polypeptide can be expressed from the nucleic acid. As such, the oncolytic virus may also be an expression vector from which the polypeptide may be expressed. Examples of such viruses are described in WO2005/049846 and WO2005/049845.

In order to effect expression of the polypeptide, nucleic acid encoding the polypeptide is preferably operably linked to a regulatory sequence, e.g. a promoter, capable of effecting transcription of the nucleic acid encoding the polypeptide. A regulatory sequence (e.g. promoter) that is operably linked to a nucleotide sequence may be located adjacent to that sequence or in close proximity such that the regulatory sequence can effect and/or control expression of a product of the nucleotide sequence. The encoded product of the nucleotide sequence may therefore be expressible from that regulatory sequence. In some preferred embodiments, the oncolytic Herpes Simplex Virus is not modified to contain nucleic acid encoding at least one copy of a polypeptide (or other nucleic acid encoded product) that is heterologous to the virus. That is the virus is not an expression vector from which a heterologous polypeptide or other nucleic acid encoded product may be expressed. Such oHSV are not suitable for, or useful in, gene therapy methods and the method of medical treatment for which they are employed may optionally be one that does not involve gene therapy.

### Administration of Herpes Simplex Virus

Administration of herpes simplex virus may involve administration at regular intervals, e.g. weekly or fortnightly. For example, doses may be given at regular, defined, intervals over a period of one of at least 1, 2, 3, 4, 5, 6, 7, 8, weeks or 1, 2, 3, 4, 5 or 6 months.

As such, multiple doses of herpes simplex virus may be administered. For example, 1, 2, 3, 4, 5, 6, 7, 8, 9, 10 or more doses of herpes simplex virus may be administered to a subject as part of a course of treatment. In some embodiments one of at least 1, 2, 3, or 4 doses of herpes simplex virus are administered to the subject, preferably at regular intervals (e.g. weekly).

Doses of herpes simplex virus may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days). The dose of herpes simplex virus given at each dosing point may be the same, but this is not essential. For example, it may be appropriate to give a higher priming dose at the first, second and/or third dosing points.

Administration of oncolytic herpes simplex virus may be of one or more treatment cycles, e.g. 1, 2, 3, 4, 5, 6, 7, 8, 9, 10 or more treatment cycles. A subject receiving multiple treatment cycles may be given subsequent treatment cycles consecutively, without a break from treatment, or may separate all or selected treatment cycles by a break from treatment, e.g. a break of 1, 2, 3, 4, 5, 6, 7, 8 or 9 days or about 1, 2, 3, or 4 weeks. Administration of oncolytic herpes simplex virus may continue until treatment fails as evidenced by tumor progression and/or unacceptable toxicity for the subject.

In some embodiments a treatment cycle may comprise, or consist of, 4 doses of oncolytic herpes simplex virus, one dose per week over a period of 4 weeks. In some embodiments a treatment cycle may comprise, or consist of, 8 doses of oncolytic herpes simplex virus, one dose per week over a period of 8 weeks. Weekly doses may be separated by 7±1 or 7±2 days. For example, weekly doses may be given on days 1, 8, 15 and 22.

In some embodiments a treatment cycle may comprise, or consist of, 4 doses of oncolytic herpes simplex virus, two doses per week over a period of 2 weeks. In some embodiments a treatment cycle may comprise, or consist of, 8 doses of oncolytic herpes simplex virus, two doses per week over a period of 4 weeks. Twice weekly doses may be separated by 4±1 or 4±2 days. For example, weekly doses may be given on days 1, 5, 8, 13 or 1, 5, 8, 12.

Subjects may receive the same dosage at each administration within a given treatment cycle, e.g. a dosage of 1×10⁷ iu or 1×10⁸ iu, or between 1×10⁸ and 1×10⁸ iu or between 1×10⁷ iu and 1×10⁸ iu. In some embodiments the first 1, 2 or 3 treatment cycles may comprise administration of a lower dosage amount at each administration, e.g. 1×10⁷ iu, and later treatment cycles may comprise administration of a higher dosage amount at each administration, e.g. 1×10⁸ iu.

Blood or serum samples may be taken at the stage of initial subject assessment (before treatment with oncolytic herpes simplex virus), and during a or each treatment cycle, e.g. on days 1, 8, 15, 22, for weekly administration, days 1, 5, 8, 13, or days 1, 5, 8, 12 for twice weekly administration. Blood or serum samples may be used to determine the presence and/or maintenance of a viral response.

Suitable dosage amounts of herpes simplex virus may be in the range 10⁵ to 10⁹ iu or 2×10⁶ to 10⁹ iu. Each dose of herpes simplex virus is preferably of greater than 1×10⁵ or 2×10⁶ iu. Each dose of virus may be in a range selected from the group consisting of: 2×10⁶ to 9×10⁶ iu, 2×10⁶ to 5×10⁶ iu, 5×10⁶ to 9×10⁶ iu, 2×10⁶ to 1×10⁷ iu, 2×10⁶ to 5×10⁷ iu, 2×10⁶ to 1×10⁸ iu, 2×10⁶ to 5×10⁸ iu, 2×10⁶ to 1×10⁹ iu, 5×10⁶ to 1×10⁷ iu, 5×10⁶ to 5×10⁷ iu, 5×10⁶ to 1×10⁸ iu, 5×10⁶ to 5×10⁸ iu, 5×10⁶ to 1×10⁹ iu, 5×10⁶ to 5×10⁹ iu, 1×10⁷ to 9×10⁷ iu, 1×10⁷ to 5×10⁷ iu, 1×10⁸ to 9×10⁸ iu, 1×10⁸ to 5×10⁸ iu. In some embodiments suitable doses may be in the range 2×10⁶ to 9×10⁶ iu, 1×10⁷ to 9×10⁷ iu, or 1×10⁸ to 9×10⁸ iu. In some embodiments suitable doses may be about 1×10⁷ iu or 1×10⁸ iu. Dosage figures may optionally be +/− half a log value.

The term ‘infectious units’ is used to refer to virus concentrations derived using the TCID50 method and ‘plaque forming units (pfus)’ to refer to plaque-based assay results. As 1 iu will form a single plaque in a titration assay, 1 iu is equivalent to 1 pfu.

In general, administration is preferably in a “effective amount”. The actual amount administered, and rate and time-course of administration, will depend on the nature and severity of the disease being treated.

Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Oncolytic herpes simplex virus may be administered by any desired route, e.g. topical, parenteral, systemic, intravenous, intra-arterial, intramuscular, intrathecal, intraocular, intratumoral, subcutaneous, oral or transdermal. In some preferred embodiments oncolytic herpes simplex virus is administered intratumorally, i.e. directly to the tumor. In such embodiments, administration by injection, which may be aided by use of imaging techniques (e.g. computer tomography, MRI) may be preferred.

Oncolytic herpes simplex virus may be administered simultaneously or sequentially with chemotherapy or radiotherapy.

Co-therapy may comprise simultaneous or sequential administration of oncolytic herpes simplex virus and chemotherapy or radiotherapy.

Simultaneous administration refers to administration of the oncolytic herpes simplex virus and chemotherapy/radiotherapy together, for example as a pharmaceutical composition containing both agents, or immediately after each other and optionally via the same route of administration, e.g. to the same tumor, artery, vein or other blood vessel.

Sequential administration refers to administration of one of the oncolytic herpes simplex virus or chemotherapy/radiotherapy followed after a given time interval by separate administration of the other agent. It is not required that the two agents are administered by the same route, although this is the case in some embodiments. The time interval may be any time interval.

Whilst simultaneous or sequential administration may be intended such that both the oncolytic herpes simplex virus and chemotherapy/radiotherapy are delivered to the same tumor tissue to effect treatment it is not essential for both agents to be present in the tumor tissue in active form at the same time.

However, in some embodiments of sequential administration the time interval is selected such that the oncolytic herpes simplex virus and chemotherapy/radiotherapy are expected to be present in the tumor tissue in active form at the same time, thereby allowing for a combined, additive or synergistic effect of the two agents in treating the tumor. In such embodiments the time interval selected may be any one of 5 minutes or less, 10 minutes or less, 15 minutes or less, 20 minutes or less, 25 minutes or less, 30 minutes or less, 45 minutes or less, 60 minutes or less, 90 minutes or less, 120 minutes or less, 180 minutes or less, 240 minutes or less, 300 minutes or less, 360 minutes or less, or 720 minutes or less, or 1 day or less, or 2 days or less.

### Chemotherapy

Chemotherapy refers to treatment of a tumor with a drug. For example, the drug may be a chemical entity, e.g. small molecule pharmaceutical, protein inhibitor (e.g. enzyme inhibitor, kinase inhibitor), or a biological agent, e.g. antibody, antibody fragment, nucleic acid or peptide aptamer, nucleic acid (e.g. DNA, RNA), peptide, polypeptide, or protein. The drug may be formulated as a pharmaceutical composition or medicament. The formulation may comprise one or more drugs (e.g. one or more active agents) together with one or more pharmaceutically acceptable diluents, excipients or carriers.

A treatment may involve administration of more than one drug. A drug may be administered alone or in combination with other treatments, either simultaneously or sequentially dependent upon the condition to be treated. For example, the chemotherapy may be a co-therapy involving administration of two drugs/agents, one or more of which may be intended to treat the tumor. In the present invention an oncolytic virus and chemotherapeutic may be administered simultaneously, separately, or sequentially which may allow the two agents to be present in the tumor requiring treatment at the same time and thereby provide a combined therapeutic effect, which may be additive or synergistic.

The chemotherapy may be administered by one or more routes of administration, e.g. parenteral, intra-arterial injection or infusion, intravenous injection or infusion, intraperitoneal, intratumoral or oral. Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

The chemotherapy may be administered according to a treatment regime. The treatment regime may be a pre-determined timetable, plan, scheme or schedule of chemotherapy administration which may be prepared by a physician or medical practitioner and may be tailored to suit the patient requiring treatment.

The treatment regime may indicate one or more of: the type of chemotherapy to administer to the patient; the dose of each drug; the time interval between administrations; the length of each treatment; the number and nature of any treatment holidays, if any etc. For a co-therapy a single treatment regime may be provided which indicates how each drug/agent is to be administered.

In some embodiments a chemotherapy agent may be an immunomodulatory agent, which may be an immune checkpoint inhibitor.

The term “immune checkpoint inhibitor” refers to molecules that totally or partially reduce, inhibit, interfere with or modulate one or more immune checkpoint proteins. An inhibitor may inhibit or block the interaction of an immune checkpoint protein with one of its ligands or receptors.

Immune checkpoint proteins negatively regulate T-cell activation or function. Numerous immune checkpoint proteins are known, such as CTLA-4 (Cytotoxic T-Lymphocyte-Associated protein 4) and its ligands CD80 and CD86; and PD-1 (Programmed Death 1) with its ligands PD-L1 and PD-L2 (Pardoll, Nature Reviews Cancer 12: 252-264, 2012), TIM-3 (T-cell Immunoglobulin domain and Mucin domain 3), LAG-3 (Lymphocyte Activation Gene-3), BTLA (CD272 or B and T Lymphocyte Attenuator), KIR (Killer-cell Immunoglobulin-like Receptor), VISTA (V-domain immunoglobulin suppressor of T-cell activation), and A2aR (Adenosine A2A receptor). These proteins are responsible for down-regulating T-cell responses. Immune checkpoint proteins regulate and maintain self-tolerance and the duration and amplitude of physiological immune responses. Immune checkpoint inhibitors include antibodies and small molecule inhibitors.

Cytotoxic T-lymphocyte associated antigen 4 (CTLA-4) is an immune checkpoint protein that down-regulates pathways of T-cell activation (Fong et al., Cancer Res. 69(2):609-5 615, 2009; Weber Cancer Immunol. Immunother, 58:823-830, 2009). CTLA-4 is a negative regulator of T-cell activation. Blockade of CTLA-4 has been shown to augment T-cell activation and proliferation. Inhibitors of CTLA-4 include anti-CTLA-4 antibodies. Anti-CTLA-4 antibodies bind to CTLA-4 and block the interaction of CTLA-4 with its ligands CD80/CD86 expressed on antigen presenting cells and thereby blocking the negative down regulation of the immune responses elicited by the interaction of these molecules. Examples of anti-CTLA-4 antibodies are described in U.S. Pat. Nos. 5,811,097; 5,811,097; 5,855,887; 6,051,227; 6,207,157; 6,682,736; 6,984,720; and 7,605,238.

Anti-CDLA-4 antibodies include tremelimumab, (ticilimumab, CP-675,206), ipilimumab (also known as IODI, MDX-DOIO; marketed under the name Yervoy™ and) a fully human monoclonal IgG antibody that binds to CTLA-4 approved for the treatment of unresectable or metastatic melanoma.

Another immune checkpoint protein is programmed cell death 1 (PD-1). PD-1, also called CD279, is a type I membrane protein encoded in humans by the PDCD1 gene. It has two ligands, PD-L1 and PD-L2. The PD-1 pathway is a key immune-inhibitory mediator of T-cell exhaustion. Blockade of this pathway can lead to T-cell activation, expansion, and enhanced effector functions. As such, PD-1 negatively regulates T cell responses. PD-1 has been identified as a marker of exhausted T cells in chronic disease states, and blockade of PD-1:PD-1L interactions has been shown to partially restore T cell function. (Sakuishi et al., JEM Vol. 207, Sep. 27, 2010, pp 2187-2194). PD-1 limits the activity of T cells in peripheral tissues at the time of an inflammatory response to infection and to limit autoimmunity. PD-1 blockade in vitro enhances T-cell proliferation and cytokine production in response to a challenge by specific antigen targets or by allogeneic cells in mixed lymphocyte reactions. A strong correlation between PD-1 expression and response was shown with blockade of PD-1 (Pardoll, Nature Reviews Cancer, 12: 252-264, 2012). PD-1 blockade can be accomplished by a variety of mechanisms including antibodies that bind PD-1 or its ligand, PD-L1, or soluble PD-1 decoy receptors (e.g. sPD-1, see Pan et al., Oncology Letters 5: 90-96, 2013). Examples of PD-1 and PD-L1 blockers are described in U.S. Pat. Nos. 7,488,802; 7,943,743; 8,008,449; 8,168,757; 8,217,149, and PCT Published Patent Application No.s: WO03042402, WO2008156712, WO2010089411, WO2010036959, WO2011066342, WO2011159877, WO2011082400, and WO2011161699.

PD-1 blockers include anti-PD-L1 antibodies and proteinaceous binding agents. Nivolumab (BMS-936558) is an anti-PD-1 antibody that was approved for the treatment of melanoma in Japan in July 2014. It is a fully human IgG4 antibody that binds to and blocks the activation of PD-1 by its ligands PD-L1 and PD-L2. Other anti-PD-L1 antibodies include lambrolizumab (pembrolizumab; MK-3475 or SCH 900475), a humanized monoclonal IgG4 antibody against PD-1; CT-011 a humanized antibody that binds PD-1. AMP-224 is a fusion protein of B7-DC; an antibody Fc portion; BMS-936559 (MDX-1105-01) for PD-L1 (B7-HI) blockade. Other anti-PD-1 antibodies are described in WO 2010/077634, WO 2006/121168, WO2008/156712 and WO2012/135408. AUNP-12 (Aurigene) is a branched 29 amino acid peptide antagonist of the interaction of PD-1 with PD-L1 or PD-L2 and has been shown to inhibit tumor growth and metastasis in preclinical models of cancer.

T cell immunoglobulin mucin 3 (TIM-3) is an immune regulator identified as being upregulated on exhausted CD8⁺ T cells (Sakuishi et al., JEM Vol. 207, Sep. 27, 2010, pp 2187-2194 and Fourcade et al., 2010, J. Exp. Med. 207:2175-86). TIM-3 was originally identified as being selectively expressed on IFN-γ-secreting Th1 and Tc1 cells. Interaction of TIM-3 with its ligand, galectin-9, triggers cell death in TIM-3′ T cells. Anti-TIM-3 antibodies are described in Ngiow et al (Cancer Res. 2011 May 15; 71(10):3540-51), and in U.S. Pat. No. 8,552,156

Other immune-checkpoint inhibitors include lymphocyte activation gene-3 (LAG-3) inhibitors, such as IMP321, a soluble Ig fusion protein (Brignone et al., 2007, J. Immunol. 179:4202-4211). Other immune-checkpoint inhibitors include B7 inhibitors, such as B7-H3 and B7-H4 inhibitors. In particular, the anti-B7-H3 antibody MGA271 (Loo et al., 2012, 5 Clin. Cancer Res. July 15 (18) 3834).

Reference to an “antibody” includes a fragment or derivative thereof, or a synthetic antibody or synthetic antibody fragment. Antibodies may be provided in isolated form or may be formulated as a medicament or pharmaceutical composition, e.g. combined with a pharmaceutically acceptable adjuvant, carrier, diluent or excipient.

In view of today's techniques in relation to monoclonal antibody technology, antibodies can be prepared to most antigens. The antigen-binding portion may be a part of an antibody (for example a Fab fragment) or a synthetic antibody fragment (for example a single chain Fv fragment [ScFv]). Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimaeric antibodies are discussed by Neuberger et al (1988, 8th International Biotechnology Symposium Part 2, 792-799).

Monoclonal antibodies (mAbs) are useful in the methods of the invention and are a homogenous population of antibodies specifically targeting a single epitope on an antigen.

Polyclonal antibodies may also be useful in the methods of the invention. Monospecific polyclonal antibodies are preferred. Suitable polyclonal antibodies can be prepared using methods well known in the art.

Fragments of antibodies, such as Fab and Fab₂ fragments may also be provided as can genetically engineered antibodies and antibody fragments. The variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sd. USA 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sd. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

By “ScFv molecules” we mean molecules wherein the V_{H} and V_{L} partner domains are covalently linked, e.g. by a flexible oligopeptide.

Fab, Fv, ScFv and dAb antibody fragments can all be expressed in and secreted from E. coli, thus allowing the facile production of large amounts of the said fragments.

Whole antibodies, and F(ab′)₂ fragments are “bivalent”. By “bivalent” we mean that the said antibodies and F(ab′)₂ fragments have two antigen combining sites. In contrast, Fab, Fv, ScFv and dAb fragments are monovalent, having only one antigen combining site. Synthetic antibodies which bind to an immune checkpoint protein may also be made using phage display technology as is well known in the art.

### Medicaments and Pharmaceutical Compositions

Viruses may be formulated as medicaments, vaccines or pharmaceutical compositions for clinical use and in such formulations may be combined with a pharmaceutically acceptable carrier, diluent or adjuvant. The composition may be formulated for topical, parenteral, systemic, intracavitary, intravenous, intra-arterial, intramuscular, intrathecal, intraocular, intratumoral, subcutaneous, oral or transdermal routes of administration which may include injection. Suitable formulations may comprise the virus in a sterile or isotonic medium. Medicaments and pharmaceutical compositions may be formulated in fluid, including gel, form. Fluid formulations may be formulated for administration by injection or via catheter to a selected region of the human or animal body.

### Cancer

A cancer may be any unwanted cell proliferation (or any disease manifesting itself by unwanted cell proliferation), neoplasm or tumor or increased risk of or predisposition to the unwanted cell proliferation, neoplasm or tumor. The cancer may be benign or malignant and may be primary or secondary (metastatic). A neoplasm or tumor may be any abnormal growth or proliferation of cells and may be located in any tissue. The cancer may optionally not be located in the central nervous system or brain. Cancers to be treated may include non-CNS solid tumor, sarcoma, chordoma, clival chordoma, peripheral nerve sheath tumor, malignant peripheral nerve sheath tumor or renal cell carcinoma.

In some embodiments the cancer may be a solid tumor. Solid tumors may, for example, be in bladder, bone, breast, eye, stomach, head and neck, germ cell, kidney, liver, lung, nervous tissue, ovary, pancreas, prostate, skin, soft-tissues, adrenal gland, nasopharynx, thyroid, retina, and uterus. Solid tumors may include melanoma, rhabdomyosarcoma, Ewing sarcoma, and neuroblastoma. The cancer may be a pediatric solid tumor, i.e. solid tumor in a child, for example osteosarcoma, chondroblastoma, chondrosarcoma, Ewing sarcoma, malignant germ cell tumor, Wilms tumor, malignant rhabdoid tumor, hepatoblastoma, hepatocellular carcinoma, neuroblastoma, melanoma, adrenocorticoid carcinoma, nasopharyngeal carcinoma, thyroid carcinoma, retinoblastoma, soft-tissue sarcoma, rhabdomyosarcoma, desmoid tumor, fibrosarcoma, liposarcoma, malignant fibrous histiocytoma, neurofibrosarcoma.

The cancer may be a sarcoma. In some embodiments the cancer is a pediatric sarcoma.

The cancer may be relapsed or refractory. The cancer may be advanced or late stage.

The cancer may be a bone cancer. The bone cancer may be a primary cancer/tumor. The bone cancer may be malignant, e.g. osteosarcoma, chondrosarcoma, Ewing's sarcoma or fibrosarcoma. The bone cancer may be a pediatric solid tumor.

The cancer may be an osteosarcoma or rhabdomyosarcoma.

The osteosarcoma may be osteoblastic, chrondoblastic, fibroblastic, mixed, high-grade surface, parosteal, periosteal, telangiectatic, or small cell osteosarcoma.

### Subject

A subject to be treated may be any animal or human. The subject is preferably human. The subject may be a human child. The subject may be male or female. The subject may be a patient. A subject may have been diagnosed with a cancer, or be suspected of having a cancer.

The subject is preferably a pediatric subject. A pediatric subject may be a human subject of age less than 18 years, or of age less than 16 years, or of age less than 14 years, or of age less than 12 years, or of age less than 10 years. The subject may optionally have a minimum age of 7 years. As such, the subject may be of age 7 to 18 years, or 7 to 16 years, or 7 to 14 years, or 7 to 12 years, or 7 to 10 years. The age may be determined at the point of first dose with oncolytic herpes simplex virus or at the point of diagnosis.

Subjects may optionally be indicated for surgical removal of tumor tissue (referred to herein as ‘tumor resection’). For example, they may have a cancer considered, by a medical practitioner, operable to remove some or all of the tumor tissue.

In such subjects, the method of treatment may comprise the direct intra-tumoral administration of oncolytic herpes simplex virus to the tumor indicated for surgical removal prior to surgery. This may be intended to stabilise tumor growth, reduce the tumor mass prior to surgery or treat portions of the tumor that are not indicated for surgical removal, e.g. metastatic lesions in other locations and/or tissues of the body. Administration of oncolytic herpes simplex virus prior to surgery may be accompanied by neoadjuvant chemotherapy or radiation therapy.

During or after surgery the oncolytic herpes simplex virus may be directly administered into tissue adjacent to or at the margin of the resected area or into tumor which could not be resected.

Subjects may be selected for treatment as being subjects who have not mounted a clinical response to previous treatment.

A subject may be immunocompetent or immunocompromised.

The subject may be seronegative for HSV-1 or HSV-2 prior to the first administration with oncolytic herpes simplex virus.

The subject may have a low lymphocyte count prior to first administration of oncolytic herpes simplex virus.

The subject may have a lymphocyte count prior to first administration of oncolytic herpes simplex virus of less than 1500, 1400, 1300, 1200, 1100, 1000, 900, 800, 700, 600, 500, 400, 300, 200 or 100 per microlitre.

### Sample

A sample may be taken from any tissue or bodily fluid of a subject. The sample may be taken from a tumor tissue or from a bodily fluid, more preferably one that circulates through the body. Accordingly, the sample may be a blood or blood-derived sample or lymph sample or lymph-derived. A blood derived sample may be a selected fraction of a patient's blood, e.g. a selected cell-containing fraction or a plasma or serum fraction. A selected cell-containing fraction may contain cell types of interest which may include white blood cells (WBC), particularly peripheral blood mononuclear cells (PBC) and/or granulocytes, and/or red blood cells (RBC).

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

### Example 1: Intratumoral Injection of HSV1716, an Oncolytic Herpes Virus, is Safe and Shows Evidence of Immune Response and Viral Replication in Young Cancer Patients

Oncolytic variants of herpes simplex virus-1 have shown anti-tumor efficacy in adults with melanoma, glioma, and other cancers. One such oncolytic HSV, HSV1716, is genetically modified to target cancer cells for viral replication and cancer cell lysis. We and others have shown HSV1716 delays tumor growth and is cytotoxic to various pediatric cancers in preclinical models. In this first evaluation of an oncolytic HSV-1 in children and young adults with cancer, we evaluated the safety and tolerability of HSV1716 administered directly by injection into tumors. HSV1716 was safe in the pediatric population with minimal toxicities noted. We also found evidence of virus replication in blood and acute inflammation on PET/CT imaging. Though no clinical responses were observed in this phase 1 trial, these findings prompt further investigation into optimal virus dosing, method of virus delivery, and combination therapies with other cancer treatments such as chemotherapy and/or immunomodulators.

Purpose:

HSV1716 is an oncolytic herpes simplex virus-1 studied in adults via injection into the brain and superficial tumors. To determine the safety of administering HSV1716 to pediatric cancer patients, we conducted a phase 1 trial of image-guided injection in young patients with relapsed or refractory extracranial cancers.

Patients and Methods:

We delivered a single dose of 105-107 infectious units of HSV1716 via computed tomography-guided intratumoral injection and measured tumor responses by imaging. Patients were eligible for up to three more doses if they achieved stable disease. We monitored HSV-1 serum titers and shedding by polymerase chain reaction and culture.

Results:

We administered a single dose of HSV1716 to eight patients and two doses to one patient. We did not observe any dose limiting toxicities. Adverse events attributed to virus included low grade fever, chills, and mild cytopenias. Six of eight HSV-1 seronegative patients at baseline showed seroconversion on day 28. Six of nine patients had detectable HSV-1 genomes by polymerase chain reaction in peripheral blood appearing on day +4 consistent with de novo virus replication. Two patients had transient focal increases in metabolic activity on 18Fluorine-deoxyglucose positron emission tomography, consistent with inflammatory reactions. In one case the same geographic region that flared later appeared necrotic on imaging. No patient had an objective response to HSV1716.

Conclusions:

Intratumoral HSV1716 is safe and well-tolerated without shedding in children and young adults with late stage, aggressive cancer. Viremia consistent with virus replication and transient inflammatory reactions hold promise for future HSV1716 studies.

### INTRODUCTION

With the recent FDA approval of the herpes simplex type 1 virus talimogene laherparepvec for melanoma by intralesional injection, oncolytic virotherapy is gaining recognition as an efficacious and safe cancer therapy. Oncolytic viruses have a large therapeutic index with limited toxic effects due to their tumor selectivity. Indeed, talimogene laherparepvec induced a 16% durable response rate as monotherapy in patients with advanced melanoma (1). HSV-1 is an attractive platform for virotherapy as it is one of the best characterized human viruses (2, 3) and its disease pathogenesis is well described (4). Diagnostic assays are standardized and practitioners have ample clinical experience dealing with HSV-1 infections. In particular, HSV is one of the few human viral pathogens for which safe and clinically proven anti-viral therapies are available. We studied a similar virus to talimogene laherparepvec, HSV1716, an oncolytic virus derived from HSV-1 strain 17. Both viruses are attenuated from their wild type counterparts by mutation in the RL1 genes encoding ICP34.5, which confers neurovirulence (5, 6). Talimogene laherparepvec is also deleted for the gene encoding ICP47, which blocks antigen presentation to major histocompatibility complex class 1 and 2 molecules, and has the coding sequence for human granulocyte-macrophage colony stimulating factor inserted in the place of ICP34.5. HSV1716 is incapable of replicating in the central nervous system (6-8), and has been extensively characterized both in vitro and in vivo. It maintains expression of thymidine kinase, targetable by administration of acyclovir, thereby providing a ‘therapeutic safety net’ in the unusual circumstance of viral replication escape and toxicity. Pre-clinically, human sarcoma and neuroblastoma cancers demonstrate replication of HSV mutants in cultured cells and human xenograft models in mice with notable anti-tumor effects (9-12). Phase 1 trials in over 80 adult cancer patients with CNS tumors, melanoma, and head and neck squamous cell carcinomas demonstrated the safety of HSV1716 with minimal toxicities (no attributable grade 3 or higher toxicities) (13-16). HSV1716 demonstrated efficacy in a phase 1 trial of adults with glioblastoma multiforme (GBM) by showing sustained responses and increased survival without additional medical intervention in 3 of 12 patients (15). One patient with GBM remained alive at last follow-up with no tumor progression 10 years after HSV1716 injection without additional medical intervention (unpublished). Herein we report the first clinical trial of HSV1716 in pediatric cancer patients. We sought to determine the safety of intratumoral injection of HSV1716 in children and young adults with non-CNS solid tumors, and to determine the dose-limiting toxicities (DLT) of intratumoral HSV1716. Our secondary aims were to assess the antiviral immune response, systemic viremia, and viral shedding after intratumoral HSV1716 injection. We also measured the antitumor activity of HSV1716 within the confines of a phase 1 trial.

### Patients and Methods

This trial received a waiver regarding the need for public discussion from the National Institutes of Health Recombinant DNA Advisory Committee. Each participating institution's local Institutional Review Board approved the trial. It was conducted under FDA Investigational New Drug BB-13196 and registered on clinical trials.gov (NCT00931931). We obtained informed consent from patients 18 years or older and/or from parents or legal guardians of patients less than 18 years of age. Child assent was obtained in accordance with local institutional policies.

### Eligibility—Inclusion Criteria

The trial population included patients with recurrent or refractory incurable non-CNS solid tumors and patients were age >7 to <30 years old at the time of virus injection. Patients were required to have a Karnofsky (age >16) or Lansky (age <16) performance score of >50%. Organ function requirements included: adequate bone marrow function (absolute neutrophil count >750/mL in absence of G-CSF for 72 hours or PEG-GCSF for 14 days, platelet count >100,000/mL and hemoglobin >9 g/dL; adequate renal function (serum creatinine <1.5× upper limit of normal for age or creatinine clearance or radioisotope GFR >70 mL/min/1.73 m2), adequate hepatic function (total bilirubin <2 times the upper limit of normal for age, alanine transaminase (ALT)<2.5× the upper limit of normal for age and albumin >2 g/dL), adequate hemostatic function (PT/INR and aPTT <1.5×ULN for age), adequate central nervous system function (baseline CNS conditions <grade 2 per CTCAE v3.0), and adequate cardiac function (shortening fraction >25% by echocardiogram, no focal wall motion abnormalities and no evidence of ischemia or significant arrhythmia on electrocardiogram). Patients with primary brain malignancies were excluded from the trial but asymptomatic patients with treated brain metastases were eligible for enrollment. We required patients to test negative for Hepatitis B surface antigen, Hepatitis C antibody, and HIV-1 and HIV-2 antibodies at or within 3 months prior to trial entry. Patients also must have fully recovered from the acute toxicities of previous therapies prior to trial enrollment. Patients could not have received myelosuppressive chemotherapy within 28 days prior to study entry or non-myelosuppressive therapy within 14 days; could not have received biologic agents within 7 days prior to trial entry; no local palliative radiation therapy within 14 days and no myeloablative radiation therapy within 42 days prior to trial entry; no immunoablative or myeloablative stem cell transplant within 6 months prior to trial entry, and no investigational agent within 28 days prior to trial entry.

Additionally, patients needed to have at least one cancer lesion amenable to HSV1716 administration by needle via imaging guidance without undue risk. The lesion(s) had to be at least 3 times greater than the volume of HSV1716 to be injected (based on available lots, the volumes were 1 mL of HSV1716 injected for dose levels 1 and 2, 5 mL for dose level 3). One lesion had to meet criteria in the first 2 dose levels and the sum total of up to 3 lesions could meet criteria in the third dose level. We recorded the longest diameter (LD) for the injected target lesion(s) as the baseline LD, which we used as reference to further characterize the objective tumor response. The response of the injected target lesion(s) determined if the patient was eligible for Part 2 of the trial in which patients could consent to receive up to 3 additional monthly doses of HSV1716. To be eligible, all injected tumors were required to be characterized as stable disease or better using a modified version of the Response Evaluation Criteria in Solid Tumors (RECIST). All measurable uninjected tumors were also identified and followed on imaging and were classified as localized or distant metastases from the site of the primary tumor.

### Eligibility—Exclusion Criteria

Exclusion criteria included a history of allogeneic stem cell transplant, currently pregnant or breast feeding, unable or unwilling to give voluntary informed consent/assent, significant infection or other severe systemic disease or medical/surgical condition deemed significant by the PI, PEG-GCSF within 14 days or G-CSF within 72 hours of trial entry, and planned use of anti-viral therapy between 2 days prior to HSV1716 administration up to 28 days after HSV1716 administration.

### Clinical Trial Design and Treatment

NCT00931931 opened as a single-center phase I trial at Cincinnati Children's Hospital Medical Center (Cincinnati, Ohio) and was subsequently expanded to include enrollment at Nationwide Children's Hospital (Columbus, Ohio). The dose escalation portion of the trial enrolled patients in a 3+3 fashion. Baseline assessments included organ function, HSV serologies and relevant imaging studies such as computed tomography (CT) and/or magnetic resonance imaging (MRI) and 18Fluorinedeoxyglucose positron emission tomography (PET)/CT imaging. All patients underwent general anesthesia to ensure safety and proper needle placement with imaging guidance. Patients received a single dose of HSV1716. Patients then recovered and were monitored in the hospital overnight for any adverse events. Peripheral blood was collected for bacterial culture, HSV PCR and culture prior to injection on Day 0 and at 1, 7, 14, 21, and 28 days after HSV1716 injection. The HSV PCR assay was our standard hospital clinical laboratory assay, which utilizes a primer for a 148 base-pair fragment for the gene encoding glycoprotein B that is present on both wild type HSV and HSV1716. Patients were discharged after the 24 hour lab draw and/or it was medically appropriate to discharge the patient home. They returned on days 4, 7, 14, 21 and 28 for labs and physical examinations to monitor AEs and organ function and immune response and virus studies. Patients were eligible for Part 2 of the trial, in which patients could receive up to three more doses after 28 days, each a minimum of 28 days apart, if they showed a tumor response in the injected lesion(s) of stable disease or better. Injection of subsequent doses required a second consent/assent. The requirement of the 28 day interval between virus doses and between patients was mandated by the FDA as a safety measure as this was the first study of an oncolytic herpes virus in children. The requirement of general anesthesia to safely administer the virus into these deep-seated tumors also limited the frequency of intratumoral virus delivery.

### Dose Limiting Toxicities

Toxicity was graded according to the NCI Common Toxicity Criteria (CTCAE) v3.0. Dose-limiting toxicity was any grade 3 or grade 4 toxicity, grade 2-4 neurologic or allergic toxicity, that was possibly, probably, or definitely attributable to participation in the study (with the exclusion of: grade 3 flu-like symptoms, grade 3 anorexia, and grade 3 pain or infection at the injection site). The highest tested and tolerated dose was predefined as the highest dose level of HSV1716 administered at which no more than 1 of 6 patients experienced a DLT.

### Evaluation of Clinical Activity

Baseline imaging was obtained within 14 days prior to the first HSV1716 dose, then again at 14 days following injection (via amendment after patient HSV03) and at 28 days, then as clinically indicated until withdrawal from the trial. All measurable lesions were deemed target lesions and were followed for response as appropriate for cancer type and location. We evaluated response according to modified Response Evaluation Criteria in Solid Tumors (RECIST) guidelines at days 14 and 28. The modification varied from RECIST v1.0 as we measured the longest diameter instead of the sum of the longest diameters.

### Virus Production, Handling, and Administration

Vials of HSV1716 were manufactured according to Good Manufacturing Practice (GMP) standards by BioReliance (Glasgow, U.K.) at either 1.0×105 (used in dose level 1) or 2.0×106 infectious units (i.u.) used in dose levels 2 (1 vial) and 3 (5 vials). Infectious units are defined as the equivalent of plaque forming units (PFU) per mL. Quality assessment HSV1716 control vials were obtained from Virttu Biologics (Glasgow, U.K.). HSV1716 was stored in an ultralow freezer (−80 C) until patient arrival. Frozen vial(s) were transported on dry ice to the interventional radiology suite, draped with a lead shield during fluoroscopy/CT scanning for needle placement, and hand thawed prior to injection through a straight needle followed by a 1 mL flush of normal saline. Thawing of HSV1716 vials required 13 minutes on average (range 5-25). Vials were checked immediately for clarity and particulate matter, sprayed and wiped down with 70% ethanol. The time elapsed from completion of the thaw to injection was 7 minutes on average. All vials contained an additional 0.1 mL of HSV1716 for quality assurance testing. Immediately following injection, vials containing residual HSV1716 were transported on ice to the lab for post-procedure virus titer assessment using the standard plaque assay procedure as previous described (17). In addition, control HSV1716 vials were thawed and assayed for quality assurance. We followed standard biosafety level 2 precautions. The acceptable range established for 10 control vials at 2×10⁶ iu was 6.3×10⁵-6.3×10⁶ iu (2 standard deviations). All post-injection titers were within the expected range (Table S1).

### Results

### Patient Characteristics

A total of 9 patients aged 8 to 30 years were enrolled and fully evaluable for safety and toxicity. Three patients were accrued to each of 3 dose levels (1×10⁵ iu, 2×10⁶ iu, and 1×10⁷ iu). Patient diagnoses included a variety of sarcomas, clival chordoma, malignant peripheral nerve sheath tumor (MPNST), and renal cell carcinoma (see Table 1). Most patients received at least two lines of therapy for relapsed or refractory disease prior to enrollment on this trial (one exception being the patient with renal cell carcinoma who was only previously treated with sunitinib). All three of the dose level 3 patients had their doses split into different needles (2 of the patients had 2 needles placed within the same tumor; HSV09 had 3 separate tumors injected).

### Serologic Responses and Toxicities

Eight of the nine patients were serologically negative for anti-HSV1 antibodies at baseline, and most patients converted following injection by day 28 (Table 2). Only HSV02 was serologically positive prior to HSV1716. No dose limiting toxicities were noted in any of the patients. Two patients had grade 3 back pain (later resolved to grade 1) related to HSV1716 and/or the intratumoral injection procedure. Grade 1 and 2 adverse events possibly or probably attributable to HSV1716 included fever, chills, and mild laboratory abnormalities such as anemia and leukopenia (Table 3). HSV09, whose dose was split into three different parenchymal lung lesions, remained hospitalized for an additional 24 hours due to monitoring of a pneumothorax, an expected complication of inserting a needle into the intrapleural space and/or pleural cavity.

Three of four patients eligible for Part 2 of the trial (more HSV1716 doses) based on stable disease of the injected lesion(s) at days +14 or +28 declined further injections due to the treating oncologist preference or concern for disease progression elsewhere. Patient HSV06 elected to receive an additional injection (denoted as “II” in Table 4), with no significant adverse events noted with either dose.

### Viremia and Virus Shedding

No viral shedding was observed in any patient on this trial as all HSV-1 cultures including blood, buccal swab, and urine at all study visits through day 28 were negative. PCR for HSV-1 genomes were also negative in all buccal swab and urine samples. Blood PCR for HSV-1 genomes were negative at baseline, day 0, and day +1 following virus injection. In contrast, blood PCR for HSV-1 genomes at day +4 turned positive in 1 patient at dose level 1, 2 patients at dose level 2, and all 3 patients at dose level 3 (6 of 9 patients total). In two patients, PCR remained positive at day +7 and in one of those patients (HSV04), it remained positive through day 28. Unfortunately, this patient's disease rapidly progressed leading to hospice care so we were unable to confirm viral clearance at a later time point.

### Disease Responses

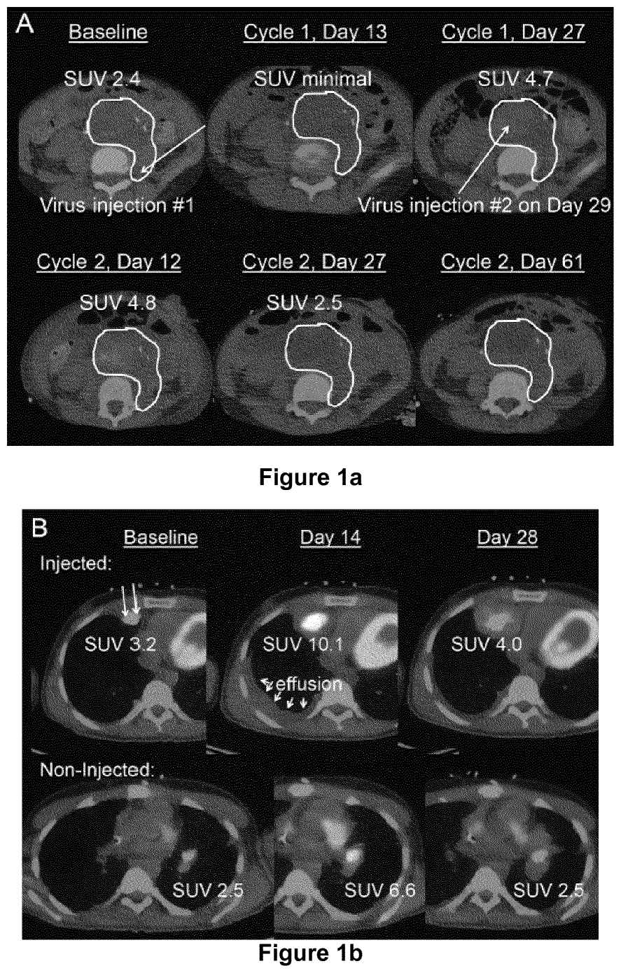

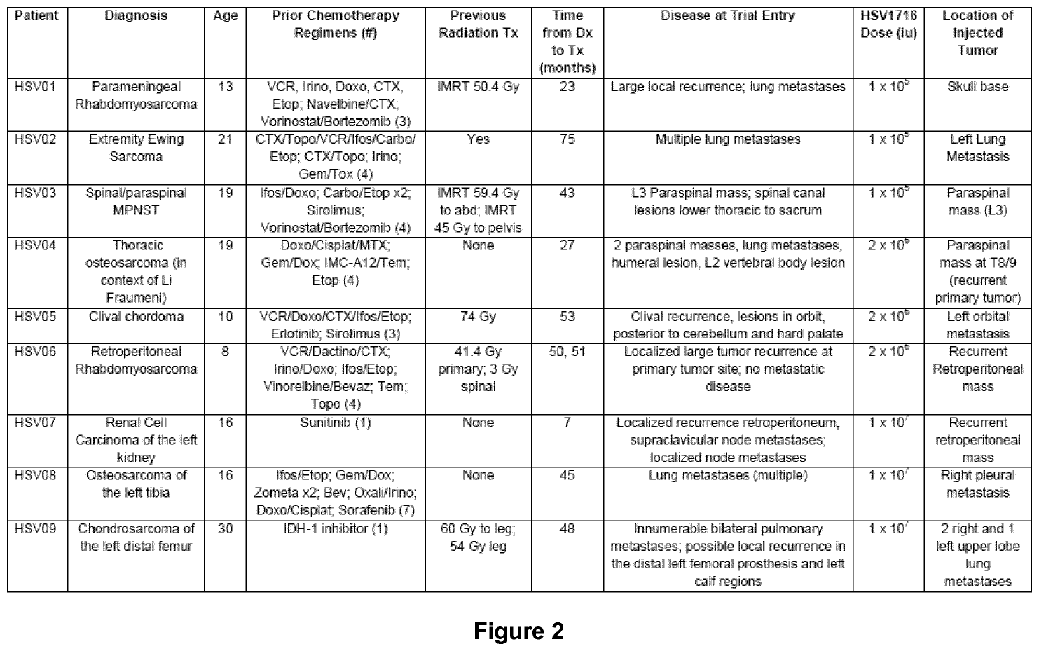

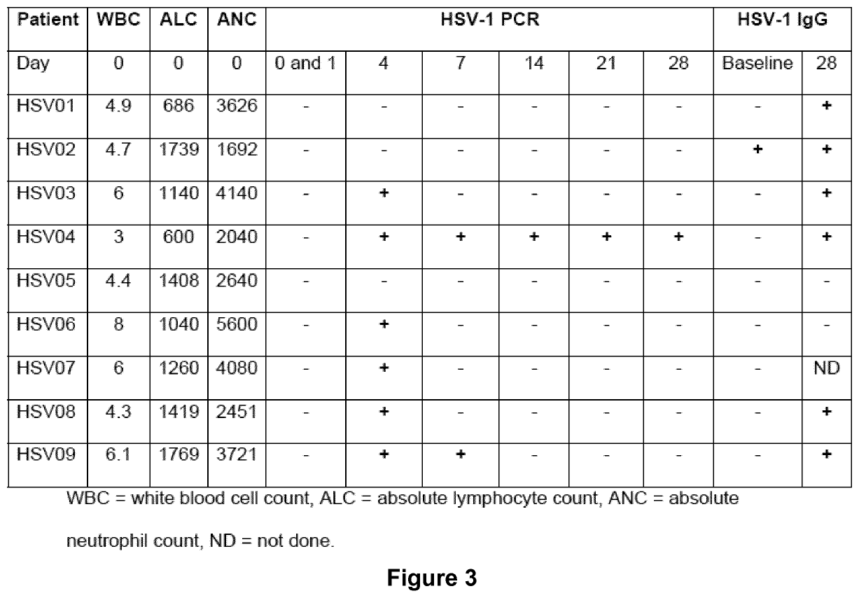

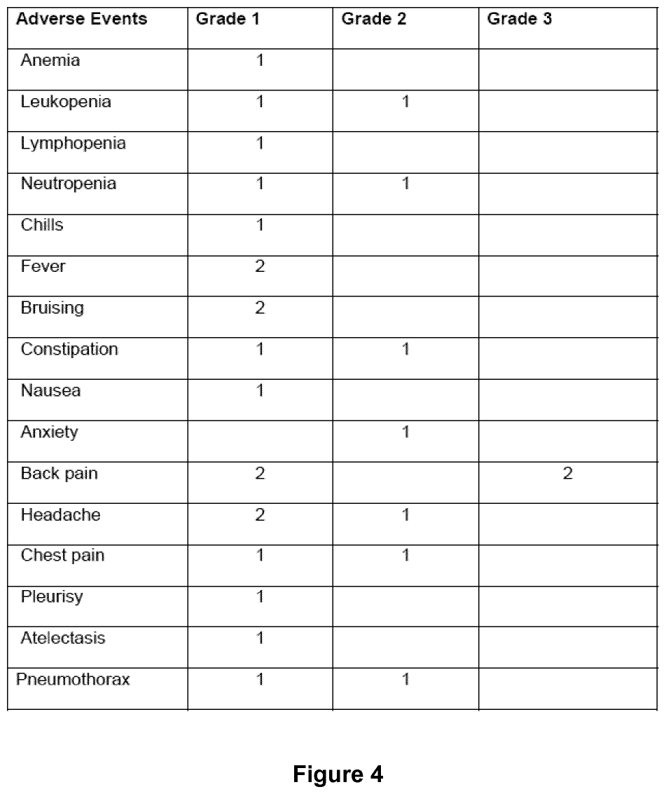

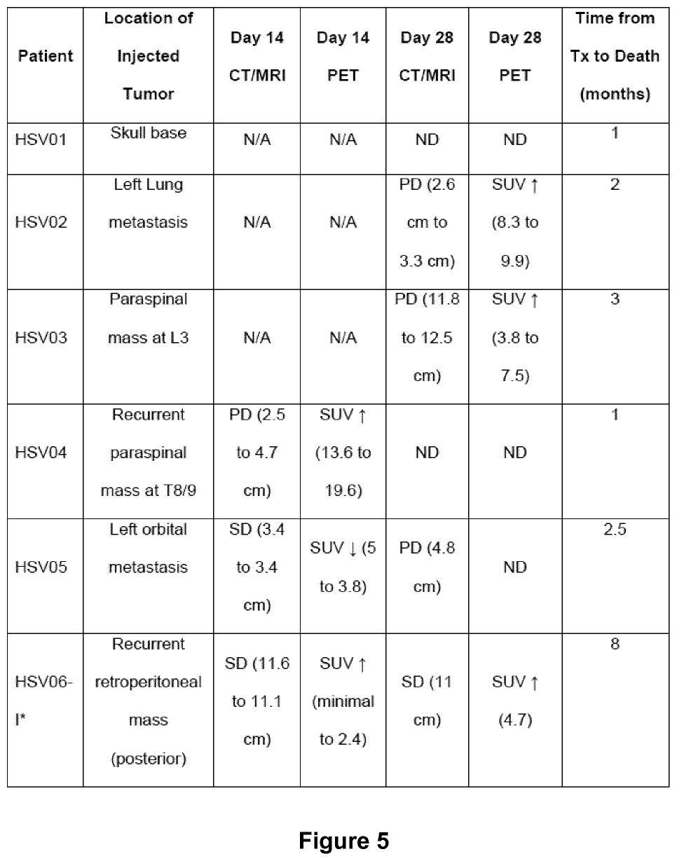

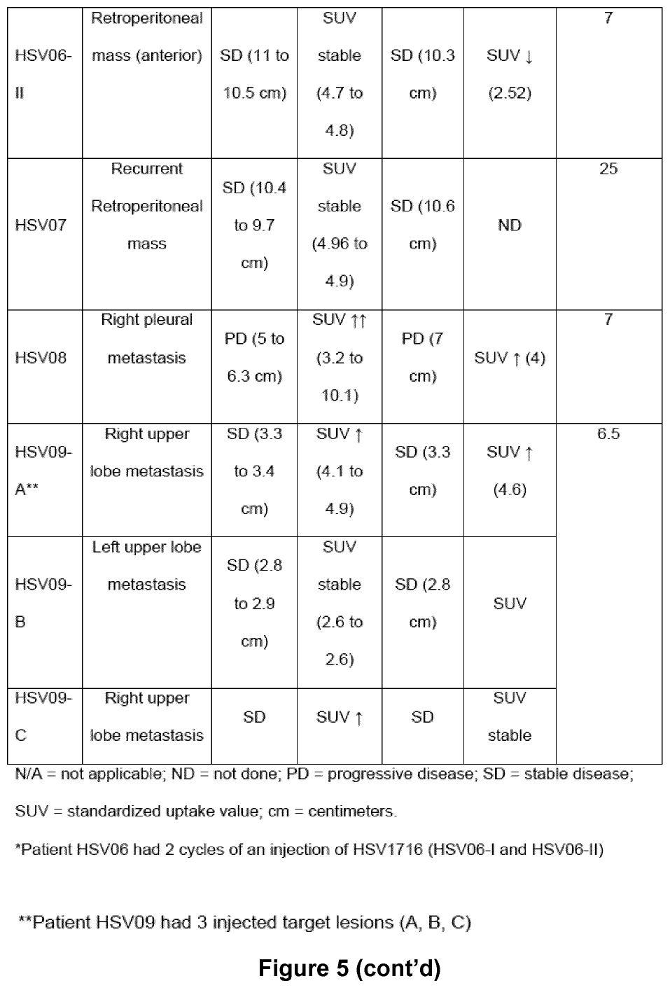

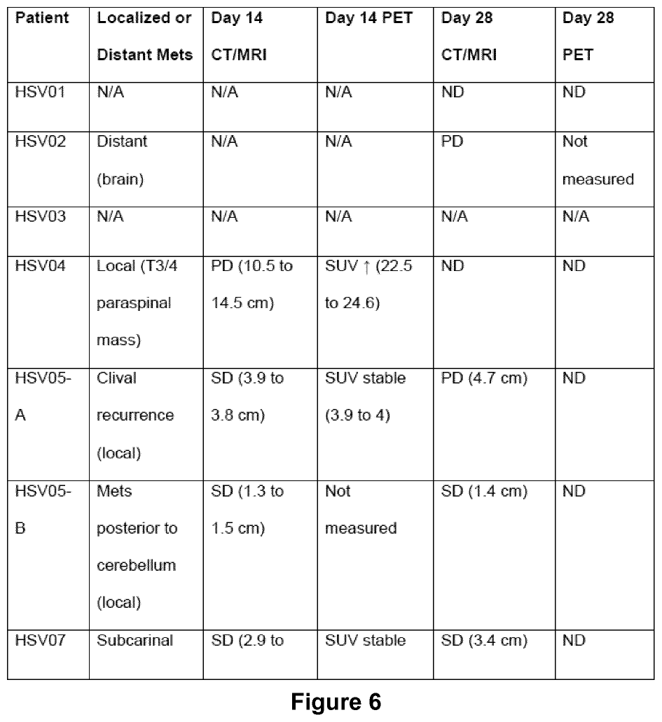

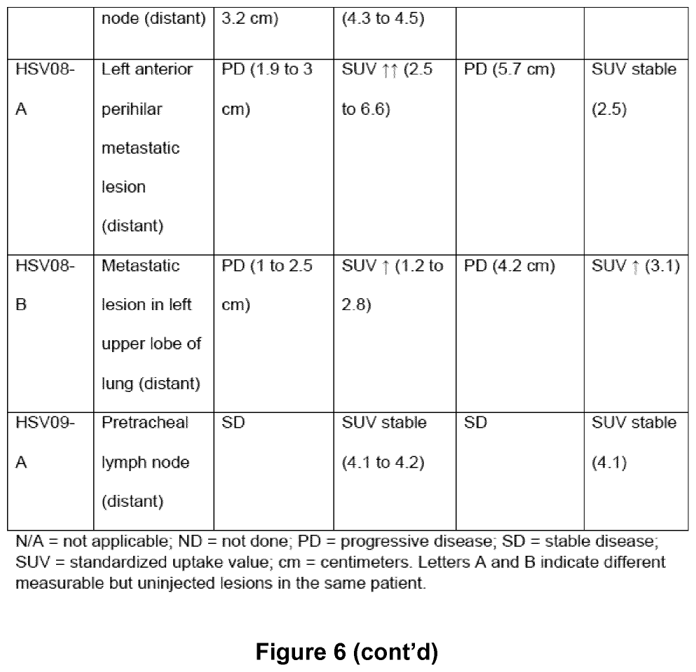

No patients had tumor shrinkage in directly injected (Table 4) or uninjected (Table 5) lesions. Four of five patients evaluated at day +14 had stable disease by cross-sectional imaging. Three of seven patients evaluated at day +28 had stable disease and one of these patients had a decrease in PET SUV (HSV09). Interestingly, in two of three patients who had multiple PET/CTs, we observed an increase in SUV either at day +14 or +28, which we initially interpreted as disease progression, followed by a spontaneous decrease back to or near baseline on subsequent images (FIG. 1). In one case, the exact geometric configuration of the increased PET signal became completely negative on subsequent scans (FIG. 1A). In another patient, we also observed a parallel flare in an uninjected metastatic tumor (FIG. 1B). As shown in Table 4, patients treated at the first 2 dose levels had a median survival of 2.25 months while the 3 patients treated at the highest dose level had a median survival of 7 months. These 3 patients also went on to other forms of therapy after discontinuing HSV1716 treatment (HSV07 received cabozantinib, HSV08 received cryoablation to the remaining tumors, and HSV09 received everolimus and pazopanib). As this is a very small number of patients all treated with different therapies after HSV1716, we are unable to draw any conclusions about the role HSV1716 may have played in their prolonged survival.

### DISCUSSION

Children with relapsed/refractory solid tumors continue to have very poor outcomes and significant toxicities from their various cancer therapies. Novel strategies and treatment modalities are urgently needed. The field of oncolytic virotherapy continues to gain momentum and offers the potential of improved outcomes with fewer toxicities for cancer patients. Based on our results, we conclude that intratumoral administration of a single dose of HSV1716 in children with relapsed/refractory non-CNS solid tumors is safe and well-tolerated. All observed adverse events that were likely attributed to virus were low grade and transient. The majority of patients enrolled in this trial were HSV-1 seronegative, suggesting that pediatric patients may benefit the most from HSV virotherapy if pre-existing anti-HSV-1 immunity is ultimately found to diminish antitumor efficacy.

Intratumoral HSV1716 resulted in systemic viremia as evidenced by initially negative and subsequent appearance of HSV-1 by PCR in the peripheral blood in most patients. The lack of a PCR signal in the peripheral blood of patients HSV01 and HSV02 may reflect that the dose used was insufficient, the location was not prone to generating viremia, or their particular tumor did not support robust virus replication. Preclinically, MPNST models show robust herpes virus replication (18), which may account for the PCR signal even with the lower dose of HSV1716 in patient HSV03. The lack of an HSV PCR signal in patient HSV05 may suggest chordoma cells do not support virus replication and/or that certain anatomic locations may not be favorable to producing viremia (i.e. a tumor in the skill base protruding into the nasal cavity and orbit). In contrast, HSV04 had a persistent PCR signal suggesting robust replication within this patient's osteosarcoma. Interestingly, HSV04 had a low ALC (600) at the time of virus injection, but in this small study it is difficult to draw any conclusions on how a low ALC may impact the ability of HSV1716 to replicate. We hypothesize the prolonged persistence of HSV detection could be due to inhibition of immune suppressor cells within the tumor such as regulatory T cells, but further research is required to determine any relationship between virus persistence and the immune microenvironment.

Most but not all patients converted their HSV-1 immune serology following virus injection. We did not observe any differences in toxicities between seronegative and seropositive patients. The reasons two of eight patients tested in this trial failed to convert to seropositive are unclear, but it is possible they had ineffective or delayed anti-viral immunity as both were heavily pretreated with chemotherapy. Though both patients had relatively normal WBC, ALC and ANC levels, the capacity of their immune systems is unknown. Further research into the functionality of the immune system at various time points in cancer treatment may be warranted to guide immunotherapy trials. As implied above regarding viremia, location of the tumor and virus injection may also play a role in seroconversion if there is limited access of immune cells to virus antigens.

Two patients notably had a transient increase in PET uptake that resolved spontaneously. The possible causes of increased glucose utilization are tumor progression or pseudoprogression, the latter from inflammation due to virus infection or stimulation of antitumor immunity. In patient HSV06, we administered a second dose at the site of uptake and, following persistence of signal 12 days later, observed complete disappearance of signal by day 27, suggesting that area of tumor was necrotic. Unfortunately, the rest of this child's large tumor mass continued to progress and the child ultimately succumbed to disease. In patient HSV08, we also observed an immediate swelling and transient increase in PET signal. The fact that the PET signal spontaneously faded suggests it was most likely consistent with an inflammatory response to virus. We do not know if the swelling, which may have been due to edema or tumor progression, would have also eventually diminished as the patient subsequently underwent cryotherapy ablation at the choice of the treating physician. The fact that an uninjected lesion also transiently flared on PET may indicate that localized HSV1716 infection had a systemic anti-tumor immune effect.

Two non-pathogenic wild type oncolytic viruses (seneca valley virus and reovirus), and one attenuated pathogenic virus (vaccinia virus), have also been studied in children and showed few toxicities but little evidence of disease response (19-21). Out of these and the current pediatric trials, this trial using HSV1716 and the trial using vaccinia virus utilized intratumoral virus administration while the other two trials used intravenous or systemic administration. The best method of virus delivery remains unclear. Thus, we are also conducting a parallel portion of this clinical trial with HSV1716 administered intravenously in pediatric patients with relapsed/refractory solid tumors. Certainly intravenous dosing is significantly less complicated due to the lack of need for sedation nor imaging guidance. A potential concern for systemic dosing is the development of anti-viral antibodies that might limit systemic delivery to tumor sites, so its use in a pediatric setting where most patients are seronegative may prove to be advantageous. Pediatric cancer patients typically enter phase 1 trials at a late stage in their disease, mostly with high tumor burdens and aggressive cancers. In contrast, patients in the Amgen trial of talimogene laherparepvec in adults had slowly growing, albeit advanced stage, melanoma. In the melanoma trial, the average time to disease response was 4 months and patients were injected with 108 infectious units of virus every 2 weeks for a minimum of 24 weeks, despite disease progression during that time (1, 22). Rather than from a direct lytic effect, the implication is that the majority of response resulted from antitumor immunity, which may take weeks to months to become robust. Thus, one rational approach to achieve enhanced benefit for pediatric cancer patients is to deliver higher and more doses of oncolytic virus than given in our trial. We plan to investigate more frequent dosing in subsequent studies, now that we have more evidence of safety with oncolytic herpes viruses as shown in this trial. The talimogene laherparepvec trial also demonstrated that higher doses of oncolytic herpes simplex virus are safe in adults by intralesional injection; however, these data were not available until near the end of our clinical trial. Thus, we only included dose-escalation to 1e7 pfu, as this was the highest dose studied in adults with HSV1716. Unlike for melanoma, however, prolonged virotherapy as a single agent may not be feasible given the rapid growth of most pediatric solid tumors. Thus, effective use of virus may require combination therapy with targeted therapies, chemotherapy or low dose radiotherapy to slow tumor growth while allowing time for virolytic or viroimmunotherapeutic effects to develop. Preclinical studies support these approaches (23-25), though concurrent therapies should be chosen and perhaps timed carefully to not interfere with virus replication (26) or the development of virus-induced antitumor immunity. Additionally, giving oncolytic virotherapy earlier in the disease course may also allow time to develop an anti-tumor immune response. Finally, herpes virotherapy may be enhanced by combination with other immune adjuncts such as T cell checkpoint inhibitors (27, 28).

In conclusion, although none of the patients had objective responses, the evidence of virus replication and inflammatory reactions we observed in pediatric cancer patients following intratumoral injection of HSV1716 are promising. We propose that using more doses of HSV1716 in addition to combination studies with other cytotoxic or cytostatic agents, radiation and/or other immunomodulators warrant further investigation. We also propose further research regarding the relationship of virus replication and the development of anti-tumor immunity in pediatric cancer to maximize the efficacy of oncolytic herpes virotherapy.

### REFERENCES

A number of publications are cited above in order to more fully describe and disclose the invention and the state of the art to which the invention pertains. Full citations for these references are provided below. The entirety of each of these references is incorporated herein.
1. Andtbacka R H, Kaufman H L, Collichio F, Amatruda T, Senzer N, Chesney J, et al. Talimogene Laherparepvec Improves Durable Response Rate in Patients With Advanced Melanoma. J Clin Oncol 2015; 33(25):2780-8 doi 10.1200/JC0.2014.58.3377.
2. Perry L J, McGeoch D J. The DNA sequences of the long repeat region and adjoining parts of the long unique region in the genome of herpes simplex virus type 1. J Gen Virol 1988; 69(Pt 11):2831-46.
3. McGeoch D J, Dalrymple M A, Davison A J, Dolan A, Frame M C, McNab D, et al. The complete DNA sequence of the long unique region in the genome of herpes simplex virus type 1. J Gen Virol 1988; 69(Pt 7):1531-74.
4. Whitley R, Gnann J. The epidemiology and clinical manifestations of herpes simplex virus infections. In: Lopez C, editor. The Human Herpesviruses. New York: Raven Press; 1993. p 69-105.
5. Ackermann M, Chou J, Sarmiento M, Lerner R A, Roizman B. Identification by antibody to a synthetic peptide of a protein specified by a diploid gene located in the terminal repeats of the L component of herpes simplex virus genome. J Virol 1986; 58(3):843-50.
6. Chou J, Kern E R, Whitley R J, Roizman B. Mapping of herpes simplex virus-1 neurovirulence to gamma 134.5, a gene nonessential for growth in culture. Science 1990; 250(4985):1262-6.
7. MacLean A R, ul-Fareed M, Robertson L, Harland J, Brown S M. Herpes simplex virus type 1 deletion variants 1714 and 1716 pinpoint neurovirulence-related sequences in Glasgow strain 17+ between immediate early gene 1 and the ‘a’ sequence. J Gen Virol 1991; 72(Pt 3):631-9.
8. Taha M Y, Brown S M, Clements G B, Graham D I. The JH2604 deletion variant of herpes simplex virus type 2 (HG52) fails to produce necrotizing encephalitis following intracranial inoculation of mice. J Gen Virol 1990; 71(Pt 7):1597-601.
9. Bharatan N S, Currier M A, Cripe T P. Differential susceptibility of pediatric sarcoma cells to oncolysis by conditionally replication-competent herpes simplex viruses. J Pediatr Hematol Oncol 2002; 24(6):447-53.
10. Parikh N S, Currier M A, Mahller Y Y, Adams L C, Di Pasquale B, Collins M H, et al. Oncolytic herpes simplex virus mutants are more efficacious than wild-type adenovirus Type 5 for the treatment of high-risk neuroblastomas in preclinical models. Pediatr Blood Cancer 2005; 44(5):469-78 doi 10.1002/pbc.20268 [doi].
11. Currier M A, Mahller Y Y, Adams L C, Cripe T P. Widespread intratumoral virus distribution with fractionated injection enables local control of large human rhabdomyosarcoma xenografts by oncolytic herpes simplex viruses. Cancer Gene Therapy 2005; 12:407-16.
12. Cinatl J, Jr., Cinatl J, Michaelis M, Kabickova H, Kotchetkov R, Vogel J-U, et al. Potent oncolytic activity of multimutated herpes simplex virus G207 in combination with vincristine against human rhabdomyosarcoma. Cancer Res 2003; 63:1508-14.
13. MacKie R M, Stewart B, Brown S M. Intralesional injection of herpes simplex virus 1716 in metastatic melanoma. Lancet 2001; 357(9255):525-6.
14. Papanastassiou V, Rampling R, Fraser M, Petty R, Hadley D, Nicoll J, et al. The potential for efficacy of the modified (ICP 34.5(−)) herpes simplex virus HSV1716 following intratumoural injection into human malignant glioma: a proof of principle study. Gene Ther 2002; 9(6):398-406.
15. Harrow S, Papanastassiou V, Harland J, Mabbs R, Petty R, Fraser M, et al. HSV1716 injection into the brain adjacent to tumour following surgical resection of high-grade glioma: safety data and long-term survival. Gene Ther 2004; 11(22):1648-58 doi 10.1038/sj.gt.3302289 3302289 [pii].
16. Mace A T, Ganly I, Soutar D S, Brown S M. Potential for efficacy of the oncolytic Herpes simplex virus 1716 in patients with oral squamous cell carcinoma. Head Neck 2008; 30(8):1045-51 doi 10.1002/hed.20840.
17. Eshun F K, Currier M A, Gillespie R A, Fitzpatrick J L, Baird W H, Cripe T P. VEGF blockade decreases the tumor uptake of systemic oncolytic herpes virus but enhances therapeutic efficacy when given after virotherapy. Gene Ther 2010; 17(7):922-9 doi gt201082 [pii] 10.1038/gt.2010.82.
18. Mahller Y Y, Rangwala F, Ratner N, Cripe T P. Malignant peripheral nerve sheath tumors with high and low Ras-GTP are permissive for oncolytic herpes simplex virus mutants. Pediatr Blood Cancer 2006; 46(7):745-54.
19. Burke M J, Ahern C, Weigel B J, Poirier J T, Rudin C M, Chen Y, et al. Phase I trial of Seneca Valley Virus (NTX-010) in children with relapsed/refractory solid tumors: a report of the Children's Oncology Group. Pediatr Blood Cancer 2015; 62(5):743-50 doi 10.1002/pbc.25269.
20. Kolb E A, Sampson V, Stabley D, Walter A, Sol-Church K, Cripe T, et al. A phase I trial and viral clearance study of reovirus (Reolysin) in children with relapsed or refractory extra-cranial solid tumors: a Children's Oncology Group Phase I Consortium report. Pediatr Blood Cancer 2015; 62(5):751-8 doi 10.1002/pbc.25464.
21. Cripe T P, Ngo M C, Geller J I, Louis C U, Currier M A, Racadio J M, et al. Phase 1 study of intratumoral Pexa-Vec (JX-594), an oncolytic and immunotherapeutic vaccinia virus, in pediatric cancer patients. Mol Ther 2015; 23(3):602-8 doi 10.1038/mt.2014.243.
22. Andtbacka R H, Ross M, Puzanov I, Milhem M, Collichio F, Delman K A, et al. Patterns of Clinical Response with Talimogene Laherparepvec (T-VEC) in Patients with Melanoma Treated in the OPTiM Phase III Clinical Trial. Ann Surg Oncol 2016 doi 10.1245/s10434-016-5286-0.
23. Toyoizumi T, Mick R, Abbas A E, Kang E H, Kaiser L R, Molnar-Kimber K L. Combined therapy with chemotherapeutic agents and herpes simplex virus type 1 ICP34.5 mutant (HSV-1716) in human non-small cell lung cancer. Hum Gene Ther 1999; 10(18):3013-29.
24. Simpson G R, Relph K, Harrington K, Melcher A, Pandha H. Cancer immunotherapy via combining oncolytic virotherapy with chemotherapy: recent advances. Oncolytic Virotherapy 2016; 5:1-13.
25. Waters A M, Stafman L L, Garner E F, Mruthyunjayappa S, Stewart J E, Friedman G K, et al. Effect of repeat dosing of engineered oncolytic herpes simplex virus on preclinical models of rhabdomyosarcoma. Translational Oncology 2016; 9(5):419-30.
26. Kulu Y, Kawasaki H, Donahue J M, Kasuya H, Cusack J, Choi E, et al. Concurrent chemotherapy inhibits herpes simplex virus 1 replication and oncolysis. Cancer Gene Ther 2013; 20(2):133-40.
27. Chen C, Wang P, Hutzen B, Haworth K, Conner J, Cripe T. Checkpoint blockade enhances oncolytic herpes virotherapy in immunosuppressive sarcoma models [abstract]. Cancer Res 2015; 76 (5 Supplement) Abstract number A14.
28. Puzanov I, Milhem M M, Minor D, Hamid O, Li A, Chen L, et al. Talimogene laherparepvec in combination with ipilimumab in previously untreated, unresectable stage IIIB-IV melanoma. J Clin Oncol 2016; 34(22):2619-26.

For standard molecular biology techniques, see Sambrook, J., Russel, D. W. Molecular Cloning, A Laboratory Manual. 3 ed. 2001, Cold Spring Harbor, N.Y.: Cold Spring Harbor Laboratory Press

1. A method of treating cancer in a human pediatric subject having a tumor, the method comprising administering an oncolytic herpes simplex virus intratumorally.

2. The method of claim 1, wherein the oncolytic herpes simplex virus is administered by intratumoral injection.

3. The method of claim 1, wherein the tumor is a solid tumor.

4. The method of claim 1, wherein the oncolytic herpes simplex virus is administered by image guided injection.

5. The method of claim 1, wherein the method of treatment comprises simultaneous, sequential or separate administration with a cytotoxic or cytostatic agent, an immunomodulatory agent, or radiation therapy.

6. The method of claim 1, wherein the method comprises determining the level of Treg cells in the subject prior to treatment with oncolytic herpes simplex virus, during a course of treatment with oncolytic herpes simplex virus and/or following conclusion of a course of treatment with oncolytic herpes simplex virus.

7. The method of claim 1, wherein the method comprises simultaneous, sequential or separate administration of an agent that supresses the regulatory T cell (Treg) response or population in the subject.

8. The method of claim 1, wherein the method comprises determining pseudoprogression of the tumor prior to treatment with oncolytic herpes simplex virus, during a course of treatment with oncolytic herpes simplex virus and/or following conclusion of a course of treatment with oncolytic herpes simplex virus.

9. A method of selecting a human subject for continued treatment with an oncolytic herpes simplex virus, the method comprising detecting a change in metabolic activity of a tumor in a human subject following administration of oncolytic herpes simplex virus to the subject, and selecting a subject in which a change is detected to receive further administration of oncolytic herpes simplex virus.

10. The method of claim 9, wherein detecting a change in metabolic activity involves detecting pseudoprogression.

11. The method of claim 9, wherein the change in metabolic activity is an increase in metabolic activity.

12. The method of claim 9, wherein the change in metabolic activity is detected by positron emission tomography.

13. The method of claim 9, wherein the administration of oncolytic herpes simplex virus to the subject is intratumoral administration.

14. The method of claim 9, wherein the administration of oncolytic herpes simplex virus to the subject is by intratumoral injection.

15. The method of claim 9, wherein the subject is a pediatric subject.

16. The method of claim 9, wherein the tumor is a solid tumor.
